(19) United States
(12) Patent Application Publication
(10) Pub. No.: US 2011/0202038 A1
(43) Pub. Date: Aug. 18, 2011

(54) GUIDEWIRE POSITIONING DEVICE

(76) Inventor: Sukhjit Gill, Oakbrook, IL (US)

(21) Appl. No.: 13/027,102

(22) Filed: Feb. 14, 2011

Related U.S. Application Data
(60) Provisional application No. 61/304,265, filed on Feb. 12, 2010.

(51) Int. Cl. A61M 25/09

(52) U.S. Cl. 604/528

(57) ABSTRACT

A guidewire positioning device and method for guiding and positioning a guidewire into a branch vessel of a main body vessel are disclosed. The device comprises a guidewire positioning tube attached to a torquable wire shaft. The distal end of the positioning tube is curved at an angle to the axis of the wire shaft. To position a guidewire into a branch body vessel, the guidewire is inserted into a main body vessel and the guidewire positioning device is advanced over the guidewire to the location of the bifurcation. The distal end of the positioning tube is positioned to point toward the targeted branch vessel by advancing or torquing the positioning device. The guidewire is advanced through the guidewire positioning tube and the curved distal end of the guidewire tube is used to direct the guidewire to enter the targeted branch vessel.

### CROSS-REFERENCES TO RELATED APPLICATIONS

This application claims the benefit and priority of U.S. Provisional Application No. 61/304,265, filed on Feb. 12, 2010, the full disclosure of which is incorporated herein by reference.

### FIELD OF THE INVENTION

This invention relates generally to device, system and method for guiding a guidewire into a branch of a main body vessel.

### DESCRIPTION OF THE RELATED ART

Coronary artery disease is the most common cause of death in the adult population in both sexes in the United States of America. Chronic total occlusion (CTO) is the complete blockage of a blood vessel and usually has serious consequences, such as a heart attack, if not treated in a proper, safe and timely fashion. The cause of blockage could be the deposition of atheromatous plaque, old thrombus or similar other deposits in the body vessel. When these coronary lesions (CTOs) become very severe, they can either be treated by coronary intervention using catheters or coronary bypass surgery. A popular method of removal of such occlusions is by coronary intervention as it is less invasive than surgery.

Coronary intervention may include the following steps: A) coronary cannulation: A long catheter is advanced through the femoral artery near the groin area to the ostium of the coronary arteries carrying the lesions. Radiopaque contrast may be injected to identify the site of blockage in the lumens of the vasculature. B) Guidewire placement: A soft non-traumatic guidewire is advanced through the coronary catheter. This guidewire crosses the obstructive lesion and is advanced through the entire length of the vessel. This guidewire acts as a track over which further instrumentation is advanced. This step is often the pivotal point in determining the success of the intervention. The placement of the guidewire is the most challenging aspect in complicated cases for the physician. C) Stent deployment: After the wire is properly positioned, a catheter that carries a stent at its distal end over an inflatable part of this catheter is advanced over the guidewire. The stent is positioned accurately at the site of the blockage. After the stent has been properly positioned the balloon is inflated thus releasing the stent and at the same time flattening the plaque. D) Post procedure confirmation: The guidewire and catheter, which was carrying the stent, are removed and a post-procedure angiogram is done to demonstrate the success of the intervention.

Safe and successful recanalization of an occluded body vessel depends on precise positioning and insertion of the guidewire into the occluded body vessel. This involves steering the guidewire to negotiate curves and bifurcations of vessels. Some lesions are present in branches that diverge from the parent vessel at varying angles, as much as 90 degrees or more. In general, the guidewire must often pass through several bends and curves before reaching the target lesion and these obstacles need different angulation and curvature of the guide wire for successful passage. The situation becomes more challenging when occlusions are present in narrow branch body vessels that are to be accessed by advancing a soft, non-traumatic tip guide wire through a main body vessel leading to the branch vessel. In such cases, the chances of erroneous diversion of such a guidewire into an unintended location such as the subintimal space or puncturing the vessel wall are very high.

Known methods to access a targeted branch body vessel either use catheter devices that have deflectable tips or employ deflectable tip guidewires. These devices have a distal tip which is capable of being deflected by the operator to a desired angle for successful penetration into a targeted branch body vessel. Devices and methods for placing bifurcated stents at branch locations in a main body vessel also use mechanisms to deflect guidewires into side branches.

While using the deflectable tip catheters and guidewire devices to access a targeted branch body vessel, the device is inserted into a main body vessel to a suitable location near the bifurcation point and thereafter the deflectable tip of the device is used to steer a guidewire into the targeted branch body vessel.

U.S. Patent Publication No. 2009/0264980 to Mackay and U.S. Pat. No. 7,089,063 to Lesh et al. and U.S. Patent Publication No. 2009/0005755 to Keith et al. disclose catheters which employ deflectable tips for placing a guidewire into a targeted branched body vessel. As a variation, some catheters may have a flexible tip at the distal end which can be curved in any direction by the operator. For guiding a guidewire into a branched body vessel, these catheters are inserted in the main body vessel and the curved tip of the catheter is placed pointing towards target branch body vessel. Thereafter, a guidewire is advanced through the catheter and into target branch of the body vessel.

U.S. Pat. No. 5,916,194 to Jacobsen et al. discloses an apparatus for directing a guide wire from a catheter to a target branch vessel. This apparatus comprises a catheter with a guidewire lumen, a shapeable distal end of the catheter, an opening in the catheter's distal sidewalls and an expandable balloon at the distal end. The distal end of the catheter is deflectable by expanding the balloon to deflect and guide a guidewire into a target body vessel.

U.S. Patent Publication No. 2004/0116832 to Friedrich et al. describes a catheter arrangement for guiding a guidewire into a branch body vessel. The device comprises two catheters which are arranged coaxially and are movable with respect to each other. The inner catheter has a pre-bent distal tip for guiding a guidewire into a target body vessel. The tilt of the pre-bent distal tip of the inner catheter can be controlled by the axial movement of the inner catheter in the outer catheter.

U.S. Pat. No. 7,371,248 to Dapolito et al. discloses a guidewire and method for steering it through tortuous vessels. This type of guidewire comprises a hollow shaft and a core wire therein and a tubular protection element at its distal end. The shaft and the core wire control deployed and collapsed configurations of the tubular protection element. Further, the application of axial tension on the protection element creates a curvature at the distal end of the guidewire which is used for steering the guidewire through tortuous vessels.

U.S. Patent Publication No. 2006/0259009 to Murray, published PCT application WO/2006/046244A2 to Turgeman et al. and U.S. Patent Publication No. 2009/0306757 to Meyer et al. disclose apparatus for diverting a guidewire through a bifurcated passageway. The device of the Turgeman publication features an elongated hollow shaft bifurcated by a partitioning element at the distal section into separate first and second lumens, with suitable feature for deflecting a guidewire. U.S. Patent Publication No. 2009/0306757 to Meyer et al. disclosing a wiring assist device includes guidewire housing members and multiple lumen arrangement for parallel and angled orientation of the guidewires. U.S. Patent Publication No. 2006/0259009 to Murray discloses a guidewire loader catheter having two lumens attached tangentially to each other with one lumen extending beyond the other at the distal end.

The guidewire positioning devices described above may suffer from various complexities and/or constraints of operation, structure and size. Even when the operator succeeds in positioning the distal end of the guidewire into a targeted branched body vessel by any of the above stated methods, the operator may not be able to successfully and safely advance the guidewire further into the branched vessel due to problems of guidewire coiling up or slipping back of the guidewire into the main body vessel on repeated pushing. Also, multiple unsuccessful attempts of positioning and insertion of the guidewire into a target branch or sub-branch of a main body vessel may cause injuries to the patient, some of which may be fatal. Therefore, there exists the need for a system and method for targeted positioning and insertion of a soft non-traumatic guidewire into branches or sub-branches of a main body vessel.

### SUMMARY OF THE INVENTION

Described herein are devices, methods, and systems for guiding a guidewire into a branch of a main body vessel.

In one aspect, a guidewire positioning device comprises a wire shaft, wherein the wire shaft comprises a distal end, a proximal end and an elongate portion therebetween; and a guidewire tube comprising a distal end and a proximal end. The distal end of the guidewire tube is curved at an angle to the axis of the wire shaft; and the guidewire tube is appended to the wire shaft and extends from the distal end toward the proximal end of the wire shaft along a portion of the wire shaft. A torquing means is attached to the proximal end of the wire shaft to transfer rotational motion to the wire shaft.

In one aspect, the wire shaft comprises at least one wire having a solid cross section that may be helically wound. The stiffness of the wire shaft is configured to decrease from the proximal end toward the distal end by either providing a decreasing cross section of wire or decrease in diameter of the wire shaft or both and may further comprise hollow or solid sections.

In one aspect, the guidewire positioning tube is formed of a flexible polymer and may taper down in diameter toward the distal end. The distal end of the guidewire positioning tube is curved at an angle to the wire shaft that is preset during manufacture.

In one aspect, the guidewire positioning tube of the device is provided with radiopaque markers at the proximal and distal ends to facilitate tracking through vasculature.

In another aspect, a system for guiding a guidewire into a branch of a main body vessel comprises the guidewire positioning device described above and a guidewire.

In yet another aspect, a method of guiding a guidewire into branch body vessels is disclosed, comprising positioning a guidewire in a main body vessel; advancing a guidewire positioning device into the main body vessel over the guidewire; wherein, the guidewire positioning device comprises a wire shaft comprising a distal end, a proximal end and an elongate portion therebetween; and a guidewire tube comprising a distal end and a proximal end; wherein, the distal end of the guidewire tube is curved at an angle to the axis of the wire shaft; and the guidewire tube is appended to the wire shaft and extends from the distal end toward the proximal end of the wire shaft along a portion of the wire shaft; advancing the guidewire through the main body vessel towards a branch location; advancing the guidewire positioning device to place the distal end of the guidewire tube pointing toward the branch body vessel; and advancing the guidewire into the branch body vessel through the guidewire tube.

In one aspect, the method involves advancing a guidewire into a main vessel past the bifurcation leading to a branch vessel; advancing the guidewire positioning device over the guidewire to the location of the branch vessel; positioning the guidewire positioning device with the distal end pointing toward the branch vessel; withdrawing the guidewire into the guidewire positioning device, and advancing the guidewire into the branch body vessel through the guidewire positioning device.

In one aspect, the guidewire is removed from the branch body vessel with the guidewire positioning device in position and a second guidewire is advanced through the proximal end of the guidewire positioning tube to access the branch body vessel.

In another aspect, the angle of curvature of the curved distal end of the guidewire tube is varied by variably advancing the guidewire through the guidewire tube.

In yet another aspect, the method comprises advancing the guidewire positioning device over a guidewire to access a second branch body vessel.

In yet another aspect, the guidewire positioning tube comprises a longitudinal opening defined by a first portion and a second portion of the guidewire positioning tube.

This, and further aspects of the present embodiments are set forth herein.

### BRIEF DESCRIPTION OF THE DRAWINGS

FIG. 1 illustrates one embodiment of the guidewire positioning device.

FIG. 2 illustrates one embodiment of the guidewire positioning device using an extended guidewire positioning tube that facilitates guidewire exchange during a procedure.

FIG. 3A illustrates one embodiment of the wire shaft of the guidewire positioning device using a single solid core wire in cross-section.

FIG. 3B illustrates one embodiment of the wire shaft of the guidewire positioning device using multiple solid core wires in cross-section.

FIG. 4 illustrates a flow diagram illustrating one embodiment of guiding a guidewire into branch body vessels.

FIGS. 5A-5C illustrate one embodiment of guiding a guidewire into branch body vessels.

FIGS. 5D-5E illustrate one embodiment of guiding a guidewire through multiple branch body vessels.

FIG. 5F illustrates one embodiment of guiding a guidewire into branch body vessels while allowing guidewire exchange.

FIG. 6A illustrates one embodiment of a guidewire positioning tube comprising a longitudinal opening.

FIG. 6B illustrates one embodiment of a guidewire positioning tube comprising a longitudinal opening and a diagonal opening at its proximal end.

FIGS. 6C-6D illustrate two embodiments of cross-sectional views of the guidewire positioning tube.

FIG. 7 illustrates a flow diagram illustrating one embodiment of separating and replacing guidewires from the guidewire positioning tube.

### DETAILED DESCRIPTION OF THE PREFERRED EMBODIMENTS

Although the detailed description contains many specifics, these should not be construed as limiting the scope of the invention but merely as illustrating different examples and aspects of the invention. It should be appreciated that the scope of the invention includes other embodiments not discussed in herein. Various other modifications, changes and variations which will be apparent to those skilled in the art may be made in the arrangement, operation and details of the method and apparatus of the present invention disclosed herein without departing from the spirit and scope of the invention as described here.

Embodiments of the present invention relate generally to devices, systems, and methods for precise guiding and positioning of a guidewire into a branch vessel of a main body vessel. In one aspect of the present embodiments, a guidewire positioning device comprises a wire shaft and a guidewire positioning tube. The wire shaft further comprises a distal end, a proximal end and an elongated portion between the distal and the proximal ends. The elongated portion of the wire shaft comprises one or more members having a solid cross-section throughout. The guidewire positioning tube is attached longitudinally to the wire shaft and extends from the distal end of the wire shaft towards the proximal end of the guidewire positioning device. The distal end of the guidewire positioning tube is bent at an angle to the axis of the wire shaft. The angle of tilt of the distal end of the guidewire positioning tube is adjustable to a fixed value prior to use or can be varied during operation according to requirements.

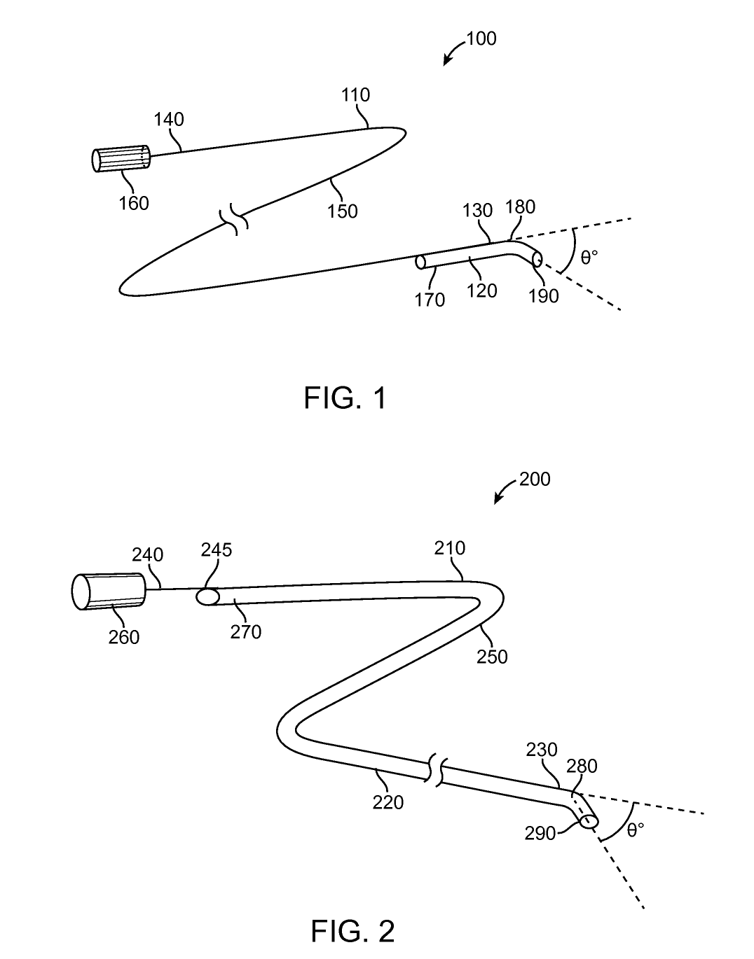

Referring now to FIG. 1, where one embodiment of the present invention is shown. As seen in FIG. 1, the guidewire positioning device 100 comprises a wire shaft 110 and a guidewire positioning tube 120. The wire shaft 110 comprises a distal end 130, a proximal end 140 and an elongated portion 150 therebetween. The wire shaft 110 may comprise a flexible wire for traversing vessels, such as tortuous and/or narrow branch body vessels. The guidewire positioning tube 120 comprises a proximal end 170 and a distal end 180 with a body disposed in between, wherein at least a portion of the guidewire positioning tube 120 is substantially longitudinally and parallelly attached to the wire shaft 110. A portion of the distal end 180 of the guidewire positioning tube 120 may extend beyond the distal end of the wire shaft 130, as is shown in FIG. 1. In one embodiment, a portion of the distal portion 180, such as a distal tip portion 190, may be highly flexible and may be configured in an unconstrained state to be curved or tilted at an angle ‘θ’ with respect to the axis of the wire shaft 110.

The guidewire positioning tube 120 may be configured for guiding a guidewire (not shown) of various configurations; for example, a guidewire with a soft, non-traumatic distal portion. Optionally, the guidewire positioning device 100 may comprise a torquing means 160 disposed on or near the proximal end of the wire shaft 110 for providing rotational torque to the wire shaft 110. Rotational torquing may be required to position the guidewire positioning tube 120 at a desired orientation in the vasculature. The torquing means 160 may be in the form of a spindle, a steering disk, a wheel, or the like that are known in the art. The torquing means 160 may be configured to distinguish between a guidewire from the guidewire positioning device 100 by tactile feedback to facilitate the operation under decreased or minimal illumination.

In one embodiment, the wire shaft 110 of the guidewire positioning device 100 may be configured as a standard length wire used in cardiac procedures, such as a wire shaft of approximately 180 cm. Alternatively, it is contemplated that the wire shaft 110 may be configured as any length or size appropriate for the operation.

In one embodiment, a portion of the guidewire positioning tube 120 may assume a tapered configuration wherein the diameter of a guidewire tube 120 is configured to decrease from a proximal portion towards the distal end. The tapered embodiment may be configured to increase the positioning accuracy of the distal tip 190 of the guidewire positioning tube 120, such configuration may be advantageous when the operating environment comprises fine vasculature.

In another embodiment, the guidewire tube 120 may comprise radiopaque markers disposed on or near the proximal tip 170 and/or the distal tip 190. The radiopaque markers may facilitate tracking the position of the guidewire tube 120, particularly, while the guidewire tube 120 navigates through narrow and/or tortuous vasculature during the operation.

The tilt angle ‘θ’ of the distal tip portion 190 of the guidewire positioning tube 120 may be configured to various degrees depending on the operation and/or the anatomy of the operating body region. In one embodiment, the tilt angle ‘θ’ may be configured as up to 45 degree with respect to the axis of the body of the guidewire position tube 120. In another embodiment, the tilt angle ‘θ’ may be configured as up to 90 degree with respect to the axis of the body of the guidewire position tube 120. In yet another embodiment, the tilt angle ‘θ’ may be configured as up to 180 degree with respect to the body of the guidewire position tube 120.

The tilt angle ‘θ’ of the distal tip portion 190 may be pre-configured by using a shape memory material or other means known in the art during manufacture, or it may be configured by the operator prior to, or during the operation. It is further contemplated that the tilt angle ‘θ’ may be variable and/or adjusted by the operator. In one embodiment, the angular orientation or curvature of the distal tip portion 190 of the guidewire positioning tube 120 may be flattened by inserting a relatively stiffer portion of a guidewire through the distal tip portion 190 of the guidewire positioning tube 120. The angular orientation may be varied as desired by withdrawing or advancing the softer portion of the guidewire through the guidewire positioning tube 120. Additionally and optionally, the distal tip portion 190 of the guidewire positioning tube 120 may be embedded with a deformable wire, such as a plastically deformable wire, to configure the desired tilt angle ‘θ’. The distal tip portion 190 may be shaped as required by the operator by deforming the deformable wire to vary the tilt angle ‘θ’.

The guidewire positioning tube 120 may be configured with elastic properties such that at least a portion of the guidewire positioning tube 120 is mutable. In one embodiment, the guidewire positioning tube 120 may be constructed of flexible material, such that the guidewire positioning tube 120 may pass through tortuous body vessels. Furthermore, the mutable configuration of the guidewire positioning tube 120 may allow the guidewire positioning tube 120 to dynamically assume various curvatures and to regain its original shape depending on the type of support positioned within. For example, a stiff tip of a guidewire may be inserted within the guidewire positioning tube 120 to provide support for a desired curvature, whereas a soft tip of a guidewire may then be positioned within the guidewire positioning tube 120 such that the tube may regain it is original configuration.

An alternative embodiment of the guidewire positioning device configured to facilitate guidewire exchange is shown in FIG. 2. The guidewire positioning device 200 comprises a wire shaft 210 and a guidewire positioning tube 220. As shown in FIG. 2, the wire shaft 210 comprises a distal end 230, a proximal end 240, and an elongated portion 250 therebetween. The guidewire positioning tube 220 comprises a proximal end 270 and a distal end 280 with a body disposed in between, the guidewire positioning tube 220 may be substantially longitudinally and parallelly attached to the wire shaft 250 and extends for a portion of the length of the wire shaft from the distal end 230 and terminates at a point 245 near the proximal end 240. The extended guidewire positioning tube 220 may be configured to enable guidewire exchange through the proximal end of tube 270 when the distal end 280 is positioned in a vessel. The distal tip portion 290 of the guidewire positioning tube 220 may be flexible and/or may be configured in an unconstrained state to be curved or tilted at an angle ‘θ’ with respect to the axis of the wire shaft 210. The curvature of the distal tip portion 290 may assume various tilted angle ‘θ’ and may be adjustable. Optionally, the guidewire positioning device 220 may comprise a torquing means 260 disposed on or near the proximal end 240 of the wire shaft 210 for providing rotational torque to the wire shaft 210 as described above.

The guidewire positioning tube 220 may be configured to extend to approximately 150 cm. In such embodiment, the guidewire positioning tube 220 is configured to enable guidewire exchange through the proximal end of the guidewire positioning tube 220 when the vasculature is accessed through the femoral artery. Alternatively, it is contemplated that the guidewire positioning tube may be configured to be of any length or size.

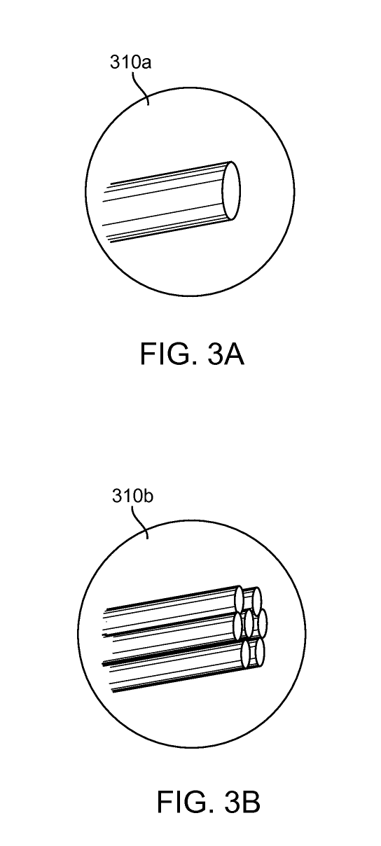

Exemplary configurations of the wire shaft are shown in FIGS. 3A-3B. As seen in FIG. 3A, the wire shaft 310 may comprise a single wire. Alternatively, as seen in FIG. 3B, the wire shaft 320 may comprise multiple wires each having a solid cross-section. The wire shaft configuration as shown in FIGS. 3A-3B may be constructed of helically wound wire or wires. Additionally, the wire shaft may be either hollow or solid in one or more portions to maintain optimum flexibility during operation. In one embodiment, a portion of the wire shaft may be configured with a decreasing diameter from the proximal end to the distal end. In another embodiment the stiffness of a portion of the wire shaft may be configured to decrease from the proximal end to the distal end. The wire shaft may be constructed of any material with suitable properties that are well known in the art for surgical applications such as stainless steel, cobalt alloy, nickel-titanium, and the like.

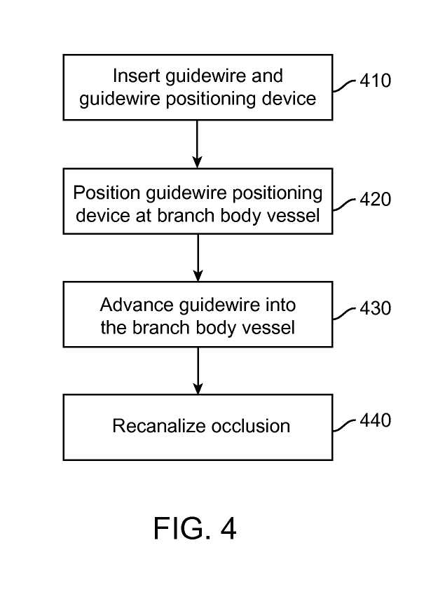

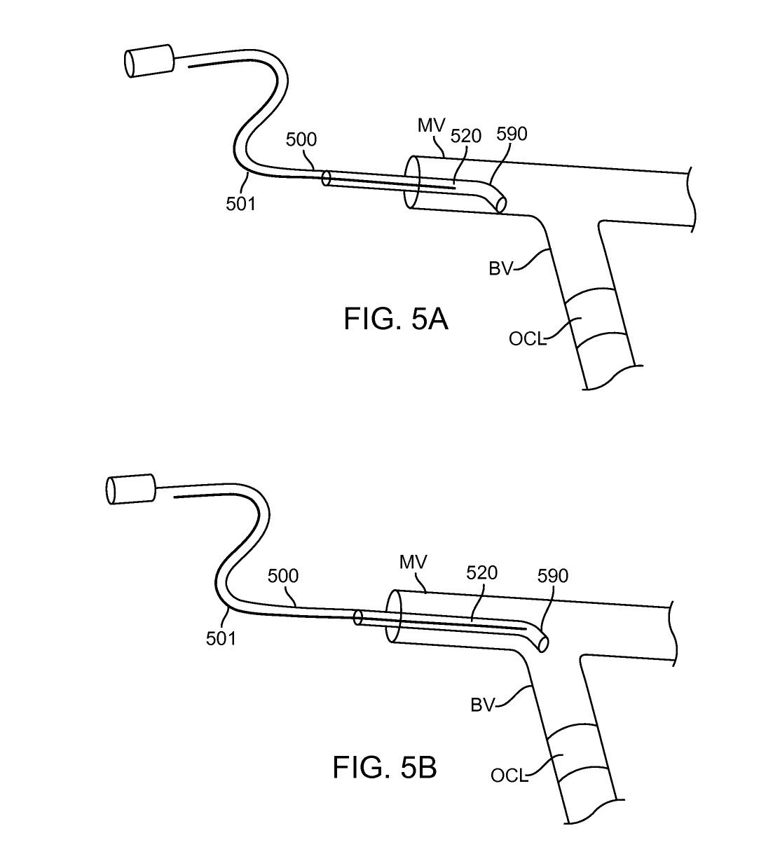

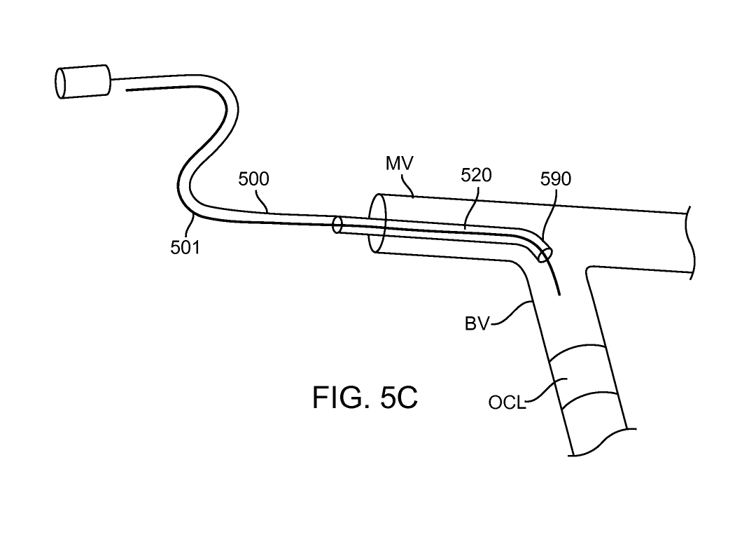

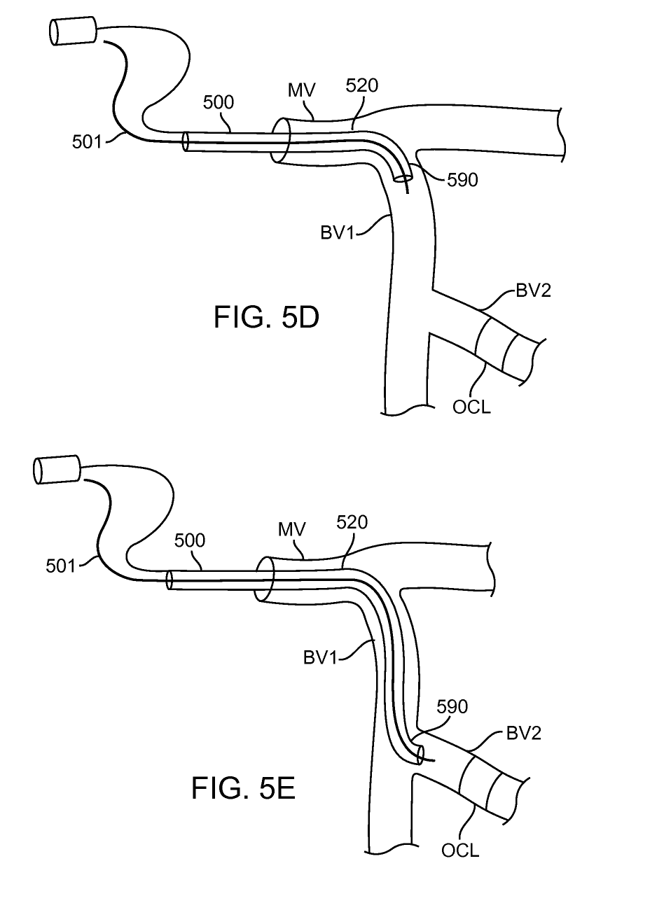

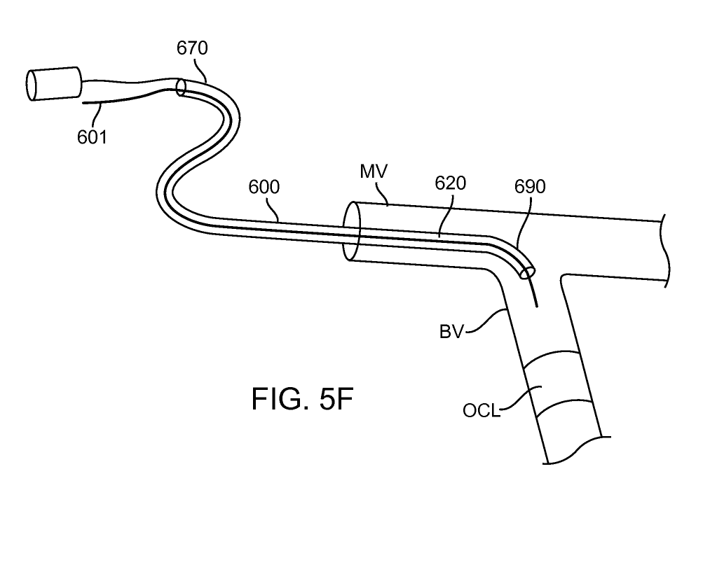

Referring now to FIG. 4, which is a flow diagram illustrating an exemplary method for positioning a guidewire in a branch body vessel. The various steps as exemplified in FIG. 4 are also illustrated in FIGS. 5A-5C. At step 410, a guidewire positioning device 500 and a guidewire 501 are inserted into the main body vessel MV as shown in FIG. 5A, by first placing a guidewire 501 in the body vessel MV and advancing the guidewire positioning device 500 over the guidewire 501. This procedure may minimize injury to the intimal layers of the body vessel MV. In one embodiment, the main body vessel MV may be the left anterior descending (LAD) or any other suitable vessel.

At step 420 and as shown in FIG. 5B, the guidewire positioning device 500 is advanced to the bifurcation in the main body vessel MV wherein a curved distal tip portion 590 of the guidewire positioning tube 520 is positioned pointing towards a branch body vessel BV. Thereafter, at step 430 and as shown in FIG. 5C, the guidewire 501 is advanced through the lumen of the guidewire positioning tube 520 such that the guidewire 501 is advanced into the branch body vessel BV. At step 440, with the guidewire 501 positioned within the branch body vessel BV, the guidewire positioning device 500 may be withdrawn and another treatment device such as a suitable catheter may be advanced over the guidewire 501. Thereafter, through the access catheter, the occlusion may be recanalized using methods well known in the art.

Additionally and optionally, it is contemplated that the embodiment of a method for positioning a guidewire in a branch body vessel may be utilized to cross multiple vessel branches in order to position the guidewire at or near the site of the occlusion. As seen in FIG. 5D, the guidewire positioning device 500 is advanced to the bifurcation in the main body vessel MV over the guidewire 501, wherein a curved distal tip portion 590 of the guidewire positioning tube 520 of is positioned pointing towards a first branch body vessel BV1. Thereafter, the guidewire 501 is advanced into the first branch body vessel BV1 and guidewire positioning device 500 is further advanced over guidewire 501 into the branch vessel BV1. The guidewire positioning device 500 may be rotated and/or otherwise manipulated to position the curved distal tip portion 590 at or near a second branch body vessel BV2, wherein the second branch body vessel BV2 is a narrow target vessel containing the occlusion OCL, shown in FIG. 5E. It is contemplated that the method as described and illustrated in FIGS. 5A-5E may be used to access narrow vasculature by advancing the guidewire positioning device 500 and the guidewire 501 one after the other in tandem. The guidewire positioning tube 520 could thus be used to support the leading portion of guidewire 501 through tortuous vasculature and across multiple bifurcations.

In an alternative embodiment of the method for guiding a guidewire into a branch body vessel, the initial steps may be substantially similar to the steps described above and illustrated in FIGS. 5A-5C, however, at the end of the procedure, as shown in FIG. 5F, with a curved distal tip portion 690 of the guidewire positioning tube 620 placed within the branch body vessel BV to be accessed, the proximal end 670 of the guidewire positioning tube 620 is configured to be accessible external to the body. As seen in FIG. 5F, guidewire 600 may be placed through the proximal end 670 of the guidewire positioning tube 620. The externally accessible proximal end 670 of the guidewire positioning tube 620 enables the original guidewire 600 to be withdrawn and a guidewire intended to penetrate occlusions can be passed through the guidewire positioning tube 620 for recanalization.

It is contemplated that the various methods described above may optionally comprise adjusting the curvature or the tilt angle ‘θ’ of the distal tip portion of the guidewire positioning tube. For example, in an embodiment where the guidewire positioning device is advanced through across multiple bifurcations as shown in FIGS. 5D-5E, the tilt angle ‘θ’ may be adjusted when the guidewire positioning device crosses the first branch. The tilt angle ‘θ’ may be adjusted such that the guidewire positioning tube may assume a substantially flat configuration to allow advancement through the first branch body vessel. Thereafter, the tilt angle ‘θ’ may be re-adjusted such that the distal tip portion of the guidewire positioning tube may assume a curved configuration wherein the distal tip portion of the guidewire positioning tube may be placed at or near the second branch body vessel. It is further contemplated that the tilt angle ‘θ’ may be adjusted throughout the operation to facilitate access and/or navigation within the body region. The adjustment of the tilt angle ‘θ’ may be accomplished by altering the degree of support as described above.

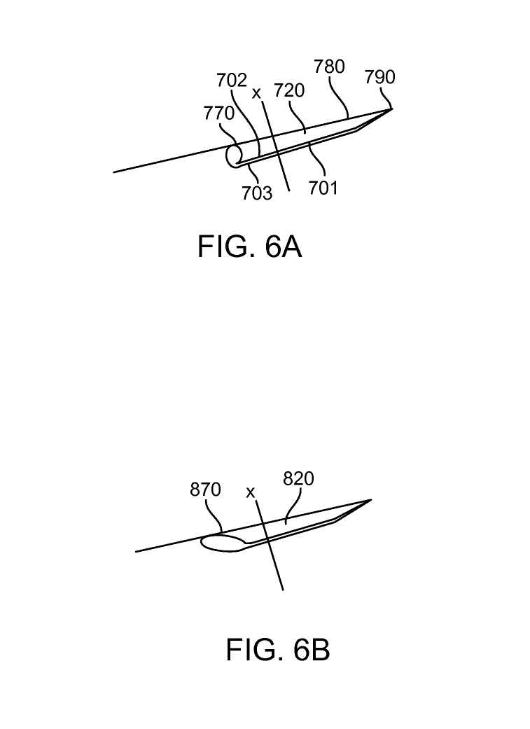

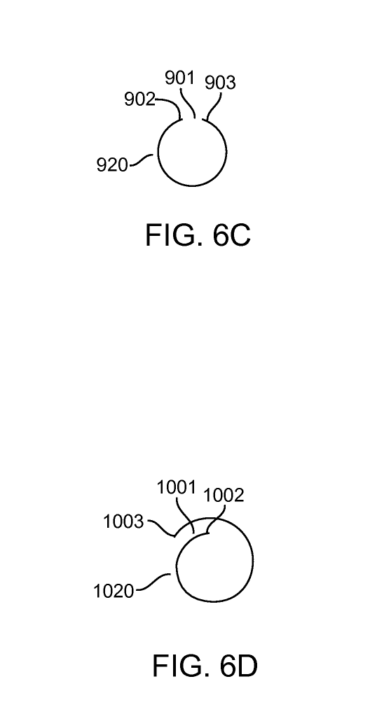

The present devices, methods, and systems further contemplate an embodiment where the guidewire positioning tube is configured to allow separation of the guidewire from the guidewire positioning tube without withdrawing the guidewire. As shown in FIG. 6A, the guidewire positioning tube 720 may comprise a first portion 702 and a second portion 703, wherein the first and the second portions 702 and 703 define a substantially longitudinal opening 701 that extends along the length of at least one side of the guidewire positioning tube 720. The longitudinal opening 701 of the guidewire positioning tube 720 is configured to accommodate the guidewire and allow the guidewire to pass through the longitudinal opening 701.

The guidewire positioning tube 720 may comprise a tapered or conical distal end 780, wherein the diameter of the guidewire positioning tube 720 decreases from a proximal portion towards the distal tip 790. This improves maneuverability and navigation of the guidewire positioning tube 720, particularly in narrow or tortuous vasculature. Alternatively, the guidewire positioning tube may comprise a substantially cylindrical or blunt distal end.

The guidewire positioning tube 720 may comprise a substantially flat opening at its proximal end 770 as shown in FIG. 6A. Alternatively, as seen in FIG. 6B, the guidewire positioning tube 820 may comprise a substantially diagonal opening at the proximal end 870. The substantially diagonal opening may allow more maneuverability and easier insertion of the guidewire.

Cross sections ‘x’ of various embodiments of the guidewire positioning tube are shown in FIGS. 6C and 6D. In one embodiment, as seen in FIG. 6C, the longitudinal opening 901 is defined by a first portion 902 and second portion 903, wherein the two portions are non-overlapping. In such embodiment, the longitudinal opening 901 may be in the form of a gap or slit on the side wall of the guidewire positioning tube 920. In another embodiment, as seen in FIG. 6D, the longitudinal opening 1001 is defined by a first portion 1002 and second portion 1003, wherein the two portions are overlapping. In such embodiment, the cross section of at least a portion of the guidewire positioning tube may assume a spiral, or folded form as seen in FIG. 6D.

Additionally and optionally, the first and/or the second portions of the various embodiments described above may be made of a flexible, elastic and/or shape-memory material such as nickel-titanium alloy. In such embodiment, when force is applied, the first and/or the second portions may be configured to alter the size and/or shape of the longitudinal opening. In an embodiment, where the guidewire positioning tube is constructed of elastic material, the opening may be configured to return to its original size or shape when the applied force is released. Furthermore, it is contemplated that the first and/or the second portions of the various embodiments may be configured to alter and to retain the altered size and/or shape of the longitudinal opening for a period of time.

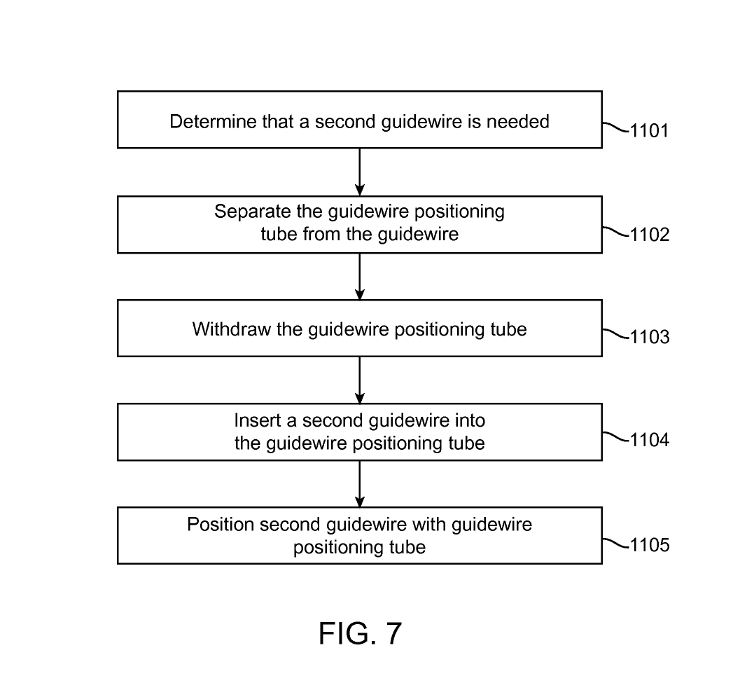

A flow diagram illustrating an exemplary method of separating the guidewire from the guidewire positioning tube 720 and positioning a new guidewire is shown in FIG. 7. In step 1101, the operator may determine that a second guidewire is needed. For example, a stiffer guidewire may be needed to penetrate an occlusion. At step 1102, the guidewire positioning tube 720 is separated from the first guidewire. The guidewire may be separated from the guidewire positioning tube 720 by passing the guidewire through the longitudinal opening 701. In one embodiment, the separation may be accomplished by moving the guidewire, the guidewire positioning tube 720 or both such that the guidewire is moved through the longitudinal opening 701. At step 1103 the guidewire positioning tube 720 is withdrawn. In one embodiment, the guidewire positioning tube 720 may be partially withdrawn from the vessel. In another embodiment, the guidewire positioning tube 720 may be fully withdrawn from the vessel. Alternatively in an embodiment where the proximal end 770 of the guidewire positioning tube 720 is configured to be accessible external to the body, guidewire positioning tube 720 may optionally not be withdrawn. At step 1104, the second guidewire is inserted into the guidewire positioning tube 720. Thereafter, at step 1105, the guidewire positioning tube 720 may be used to position the second guidewire at the desired location using the same steps as in previous methods as illustrated in FIGS. 5A-C above.

In various embodiments of the devices, systems, and methods described above, it is contemplated that various kinds of guidewires may be used. For example, the guidewire may be a stiff guidewire, an ultrasound guidewire and/or guidewires with active means for eliminating occlusions such as RF devices, laser devices and the like. Similarly, other surgical devices for treatment of vascular occlusions such as mechanical burrs can be quickly and safely positioned within narrow body lumens for recanalization procedures.

The guidewire positioning devices, systems, and the methods as described herein may minimize repeated attempts of successfully guiding a guidewire into a target body vessel, prevent guidewire slippage and coil formation during the process and assist in safely directing a soft non-traumatic guidewire into a into a targeted body vessel. The devices and methods disclosed herein may also enable rapid and safe procedures to be carried out, while increasing patient comfort and reducing fatigue to the surgeon in performing complicated procedures.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A guidewire positioning system for guiding a guidewire, comprising:
a wire shaft comprising a distal end, a proximal end and an elongate portion therebetween; and
a guidewire tube comprising a distal end and a proximal end;
wherein a portion of the distal end of the guidewire tube is curved at an angle with respect to the axis of the wire shaft, and wherein the guidewire tube is appended to the wire shaft to extend along a portion of the wire shaft.

2. The system of claim 1, comprising a torquing means at the proximal end of the wire shaft to transfer torque to the wire shaft.

3. The system of claim 1, wherein a portion of the guidewire tube decreases in diameter from the proximal to the distal end.

4. The system of claim 1, wherein a portion of the guidewire tube is formed of a flexible polymer.

5. The system of claim 1, wherein the guidewire comprises one or more radiopaque markers at its distal and proximal ends.

6. The system of claim 1, wherein the curved distal end of the guidewire tube is reinforced with a shapeable wire.

7. The system of claim 1, wherein the guidewire tube comprises a longitudinal opening along a side of the guidewire tube that extends from the proximal end of the guidewire tube to the distal end.

8. The system of claim 7, wherein the longitudinal opening is defined by a first and second portion of the guidewire tube.

9. The system of claim 8, wherein the two portions are configured to overlap.

10. The system of claim 8, wherein the portions are elastically separable.

11. The system of claim 1, further comprising a guidewire, wherein the guidewire is configured to access a body vessel through the guidewire tube.

12. The system of claim 11, further comprising a torquing means at the proximal end of the guidewire to transfer torque to the guidewire.

13. The system of claim 11, wherein the guidewire has a soft, flexible tip.

14. The system of claim 11, wherein the angle of curvature of the curved distal end of the guidewire tube is variable by variably advancing the guidewire through the guidewire tube.

15. The system of claim 11, wherein the guidewire tube comprises a longitudinal opening along a side of the guidewire tube that extends from the proximal end of the guidewire tube to the distal end.

16. A method of guiding a guidewire into branch body vessels, comprising:
positioning a guidewire in a main body vessel;
advancing a guidewire positioning device into the main body vessel over the guidewire, wherein the guidewire positioning device comprises a curved distal end configured to be at an angle with respect to the axis of a body of the guidewire positioning device;
advancing the guidewire through the main body vessel towards a branch location;
placing the curved distal end of the guidewire positioning device at a portion of a branch body vessel; and
guiding the guidewire into the branch body vessel through the guidewire positioning device.

17. The method of claim 16, wherein the guidewire is advanced into the main vessel past the bifurcation leading to the branch vessel, and further comprising:
advancing the guidewire positioning device over the guidewire to the location of the branch vessel;
positioning the guidewire positioning device with the distal end pointing toward the branch vessel;
withdrawing the guidewire into the guidewire positioning device; and
advancing the guidewire into the branch body vessel through the guidewire positioning device.

18. The method of claim 16, further comprising:
removing the guidewire from the branch body vessel with the guidewire positioning device in position; and
advancing a second guidewire through the proximal end of the guidewire positioning tube to access the branch body vessel.

19. The method of claim 16, further comprising adjusting the angle of curvature of the curved distal end of the guidewire tube by variably advancing the guidewire through the guidewire tube.

20. The method of claim 16, comprising further advancing the guidewire positioning device over the guidewire to access a second branch body vessel.
